(19) United States
(12) Patent Application Publication
(10) Pub. No.: US 2013/0165750 A1
(43) Pub. Date: Jun. 27, 2013

(54) ENDOSCOPE CAPABLE OF CHANGING DIRECTION

(71) Applicant: Yi-Chen Chou, Tainan City (TW)

(72) Inventor: Yi-Chen Chou, Tainan City (TW)

(73) Assignee: HIMAX IMAGING LIMITED, Tainan City (TW)

(21) Appl. No.: 13/335,744

(22) Filed: Dec. 22, 2011

(51) Int. Cl. A61B 1/04; A61B 1/00

(52) U.S. Cl. 600/109; 600/146

(57) ABSTRACT

The invention is directed to an endoscope having a distal section coupled to a distal end of a tube, and a wire disposed in the tube, wherein a distal end of the wire is fixed at a distal point of the tube. The endoscope also includes a handling section coupled to a proximal end of the tube. The wire slidingly passes through a sliding point that is situated between the distal point and the proximal end of the wire, such that the wire is constrained at the sliding point. A proximal end of the wire is capable of being pulled to bend the tube.

### BACKGROUND OF THE INVENTION

1. Field of the Invention

The present invention generally relates to an endoscope, and more particularly to an endoscope capable of being bent or rotated.

2. Description of Related Art

An endoscope is an instrument that is capable of being inserted into an organ to examine the interior of the organ. The endoscope generally includes a flexible tube; a light source disposed at a proximal end of the endoscope and the light is delivered via an optical fiber; and a lens system disposed at a distal end. of the en.doscope for collectin.g image that is transferred via an optical fiber.

Due to the miniature dimension of the endoscope, the manufacturing of the lens system requires great effort and thus making the overall cost high and accessibility low. Moreover, infection may probably occur due to inadequate cleaning of the endoscope. As far as the cost and infection are concerned, a single-use disposable endoscope may alleviate the problems mentioned above.

For the reason that the conventional endoscope is either high-priced or at the risk of infection, a need has arisen to propose a novel endoscope that eliminates the problems mentioned above.

### SUMMARY OF THE INVENTION

In view of the foregoing, the embodiment of the present invention provides an endoscope having a distal section made of a wafer-level imaging module, capable of changing direction. The endoscope of the embodiment may be adaptable to disposable use to eliminate possibility of infection.

According to one embodiment, an endoscope includes a tube, a distal section, a wire and a handling section. The distal section is coupled to a distal end of the tube, and a wire is disposed in the tube, wherein a distal end of the wire is fixed at a distal point of the tube. The handling section is coupled to a proximal end of the tube. The wire slidingly passes through a sliding point that is situated between the distal point and the proximal end of the wire, such that the wire is constrained at the sliding point. A proximal end of the wire is capable of being pulled to bend the tube.

### BRIEF DESCRIPTION OF THE DRAWINGS

FIG. 1A schematically shows a lateral cross-sectional view of an endoscope according to one embodiment of the present invention;

FIG. 1B shows a cross-sectional view along a section line 1B-1B′ of FIG. 1A;

FIG. 2 schematically shows a perspective view of the WLM of FIG. 1;

FIG. 3A schematically shows a cross-sectional view of a portion of the tube before the rotation knob has been rotated;

FIG. 3B schematically shows a cross-sectional view of a portion of the tube after the rotation knob has been rotated; and

FIG. 4 schematically shows a perspective view of the tube according to a specific embodiment of the present invention.

### DETAILED DESCRIPTION OF THE INVENTION

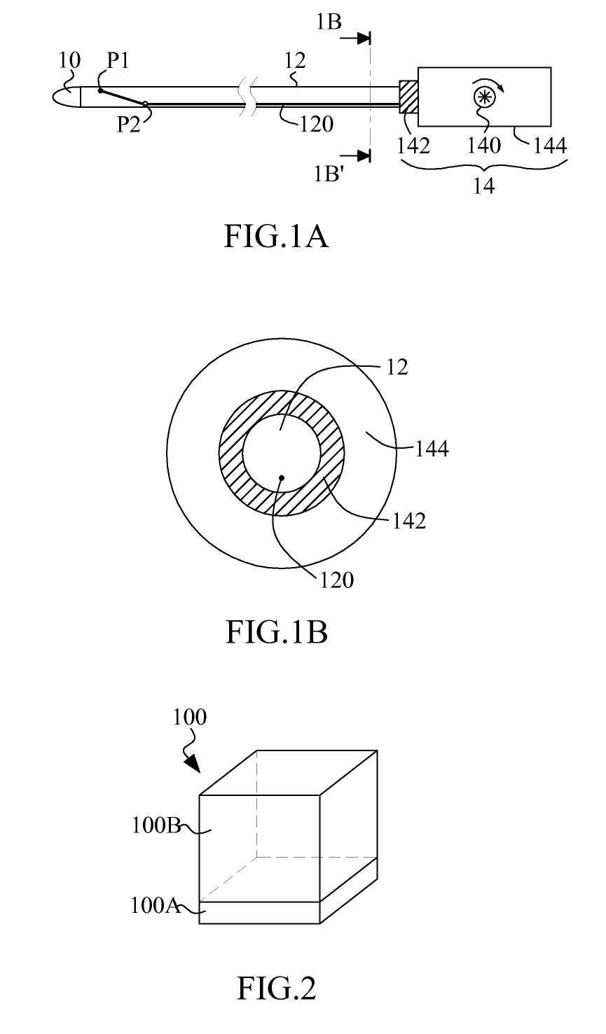

FIG. 1A schematically shows a lateral cross-sectional view of an endoscope according to one embodiment of the present invention. In the embodiment, the endoscope includes a distal section 10, a tube 12 and a handling section 14. Specifically, the distal section 10 is disposed at and coupled to a distal end of the tube 12. The distal section 10 of the embodiment primarily includes a wafer-level imaging module (or wafer-level module, WLM, for short) 100. FIG. 2 schematically shows a perspective view of the WLM 100. In the embodiment, the WLM 100 includes a wafer-level image sensor 100A and wafer-level optics (WLO) 100B. The wafer-level image sensor 100A is situated facing the distal end of the tube 12, and may be, but not limited to, a complementary metal oxide semiconductor (CMOS) image sensor (commonly abbreviated as CIS). The wafer-level optics 100B, such as a lens, is situated away from the distal end of the tube 12, and may be made of, but not limited to, glass. The wafer-level image sensor 100A and the wafer-level optics 100B may be bonded together, for example, with an adhesive. Compared to the conventional endoscope, the endoscope of the present embodiment makes use of the mass-productivity and low cost of semiconductor technique to manufacture the imaging system of the endoscope, and is adaptable to the disposable endoscope to eliminate possibility of infection. Wafer-level (or wafer-scale) module is a technique of fabricating miniaturized optics such as lens module or camera module at the wafer level using semiconductor techniques, and details of manufacturing the wafer-level imaging module 12 may be referred, for example, to U.S. Pat. No. 7,564,496 to Wolterink et al., entitled “Camera device, method of manufacturing a camera device, wafer scale package,” the disclosure of which is incorporated herein by reference.

In the embodiment, a wire 120 is disposed in the tube 12. The distal end of the wire 120 is fixed at a distal point P1, for example, on the inner surface of the tube 12. The proximal end of the wire 120 is coupled to the handling section 14. The wire 120 slidingly passes through a sliding point P2 that is situated between the distal point P1 (e.g., the distal end of the wire 120) and the proximal end of the wire 120. The wire 120 is constrained (but not fixed) at the sliding point P2, which acts as a pivot point while bending the tube 12.

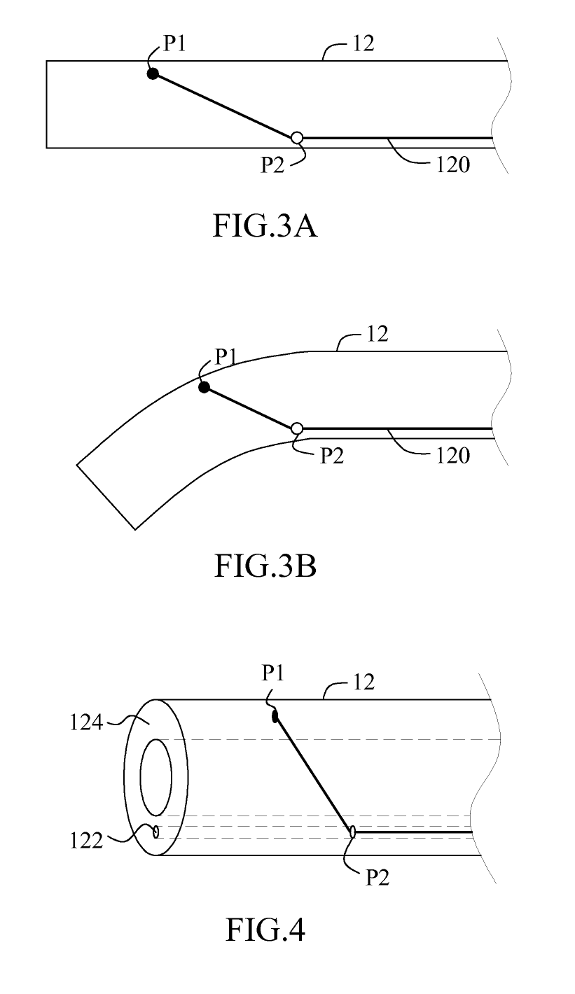

According to one aspect of the embodiment, as shown in FIG. 1A, the proximal end of the wire 120 is fastened to a rotation knob 140. When the rotation knob 140 rotates, for example, clockwise, the wire 120 is pulled toward the proximal end of the endoscope. FIG. 3A schematically shows a cross-sectional view of a portion of the tube 12 before the rotation knob 140 has been rotated, and FIG. 3B schematically shows a cross-sectional view of the same portion of the tube 12 after the rotation knob 140 has been rotated, thereby pulling the wire 120 toward the proximal end of the endoscope. As the wire 120 is pulled (by the rotation knob 140), the length between the fixed distal point P1 and the sliding point P2 is reduced. The shortened portion of the wire 120 between P1 and P2 therefore bends the distal portion (i.e., the portion being away from the sliding point P2 and toward the distal end) of the tube 12. Although the rotation knob 140 is exemplified in the embodiment, it is appreciated by a person skilled in the art that a scheme other than the rotation knob 140 may be employed to pull the wire 120.

According to another aspect of the embodiment, the handling section 14 further includes a rotation ring 142 that is disposed between the tube 12 and a stationary portion 144. One end of the rotation ring 142 is fixed to the proximal end of the tube 12, and the other end of the rotation ring 142 is rotationally coupled (but not fixed) to the stationary portion 144. The axis of the rotation ring 142 coincides with the axis of the tube 12. FIG. 1B shows a cross-sectional view along a section line 1B-1B′ of FIG. 1A. Accordingly, as the rotation ring 142 rotates, the tube 12 follows the rotation ring 142 to rotate as well while the stationary portion 144 keeps holding. Although the rotation ring 142 is exemplified in the embodiment, it is appreciated by a person skilled in the art that a scheme other than the rotation ring 142 may be employed to rotate the tube 12.

FIG. 4 schematically shows a perspective view of the tube 12 according to a specific embodiment of the present invention. In the embodiment, the tube 12 has a passage channel 122 formed within a cladding layer 124 of the tube 12. The wire 120 is disposed in the passage channel 122, except that the portion between the distal point P1 and the sliding point P2 is disposed in the hollow core defined, by the cladding layer 124 of the tube 12. The distal point P1 is fixed at the cladding layer 124, and may, for example, be distanced from the sliding point P2 with a predetermined longitudinal (i.e., along the length of the tube 12) distance, and may be situated opposite the sliding point P2. As described above, the wire 120 is constrained (but not fixed) at the sliding point P2, which acts as a pivot point while bending the tube 12.

Although specific embodiments have been illustrated and described, it will be appreciated by those skilled in the art that various modifications may be made without departing from the scope of the present invention, which is intended to be limited solely by the appended claims.

What is claimed is:

1. An endoscope, comprising:
a tube;
a distal section coupled to a distal end of the tube;
a wire disposed in the tube, wherein a distal end of the wire is fixed at a distal point of the tube; and
a handling section coupled to a proximal end of the tube;
wherein the wire slidingly passes through a sliding point that is situated between the distal point and the proximal end of the wire, such that the wire is constrained at the sliding point; and
wherein a proximal end of the wire is capable of being pulled to bend the tube.

2. The endoscope of claim 1, wherein the distal section comprises:
a wafer-level image sensor; and
a wafer-level optics bonded with the wafer-level image sensor;
wherein the wafer-level image sensor is situated facing the distal end of the tube, and the wafer-level optics is situated away from the distal end of the tube.

3. The endoscope of claim 2, wherein the wafer-level image sensor comprises a complementary metal oxide semiconductor (CMOS) image sensor.

4. The endoscope of claim 2, wherein the wafer-level optics comprises a lens.

5. The endoscope of claim 1, further comprising means for pulling the wire.

6. The endoscope of claim 5, wherein the pulling means comprises a rotation knob, to which the proximal end of the wire is fastened, wherein the rotation knob is disposed in the handling section.

7. The endoscope of claim 6, wherein the handling section further comprises a stationary portion, in which the rotation knob is disposed.

8. The endoscope of claim 1, further comprising means for rotating the tube.

9. The endoscope of claim 8, wherein the rotating means comprises a rotation ring disposed between the tube and a stationary portion of the handling section, wherein one end of the rotation ring is fixed to the proximal end of the tube, and another end of the rotation ring is rotationally coupled to the stationary portion.

10. The endoscope of claim 1, wherein the tube comprises a cladding layer that defines a hollow core in the tube.

11. The endoscope of claim 10, wherein, the tube has a passage channel formed within the cladding layer.

12. The endoscope of claim 11, wherein the wire is disposed in the passage channel, except that a portion between the distal point and the sliding point is disposed in the hollow core.

13. The endoscope of claim 1, wherein the distal point is situated opposite the sliding point.
